(19) United States
(12) Patent Application Publication
(10) Pub. No.: US 2014/0261447 A1
(43) Pub. Date: Sep. 18, 2014

(54) Method and Apparatus Pertaining to the Warming of a Patient

(71) Applicant: MEDLINE INDUSTRIES, INC., Mundelein, IL (US)

(72) Inventor: Andrew Giles, Libertyville, IL (US)

(73) Assignee: MEDLINE INDUSTRIES, INC., Mundelein, IL (US)

(21) Appl. No.: 13/832,397

(22) Filed: Mar. 15, 2013

(51) Int. Cl. A61F 7/08; A61G 13/12

(52) U.S. Cl. CPC A61F 7/08; A61G 13/12
USPC 128/845

(57) ABSTRACT

A patient warming apparatus includes a compressible patient support pad and an electrically-resistive heating film disposed in conjunction with the compressible patient support pad that is configured to provide heat to at least a portion of a patient that is supported atop the compressible patient support pad. By one approach the electrically-resistive heating film comprises a substantially non-resilient, dead-foldable film substrate. By one approach the substantially non-resilient, dead-foldable film substrate comprises a polyimide cloth-like film having a conductive coating permanently adhered to one side thereof. This conductive coating can form two physically-separated electrodes. By one approach these electrodes need not be electrically coupled by conductive traces.

### TECHNICAL FIELD

This invention relates generally to patient warming and more particularly to patient warming via patient support surfaces.

### BACKGROUND

Providing local warming to a patient during, for example, surgery comprises a known area of endeavor. While sometimes employed to improve the patient's comfort, patient warming can also provide important patient treatment and/or recovery support as well.

In addition to passive mechanisms (such as blankets to dispose over a patient) active mechanisms are sometimes used to provide local, controlled heating to portions of a patient's body. Such active mechanisms include the use of, for example, electric heating elements that convert electricity into heat. One illustrative approach in these regards employs a laminate assembly comprising a Mylar substrate having carbon ink resistors formed thereon and silver ink traces to form needed connections. Metallic copper strips then overlay the foregoing. In a typical application setting a patient support pad has such an electric heating element disposed thereon or, more typically, therein. Upon applying electrical power to the electric heating element the resistors convert some of that electricity to heat that in turn warms a patient lying atop the patient support pad.

Though often a successful approach to patient warming, such electric heating elements are often unduly frangible and fail during use. The Mylar substrate, for example, typically comprises a highly resilient material with poor dead-folding characteristics. In turn, bending of the substrate can lead to delamination and consequent failure of part or all of the heating element. Unfortunately, such physical challenges to the integrity of such an assembly can and do happen in the context of providing medical services.

### BRIEF DESCRIPTION OF THE DRAWINGS

The above needs are at least partially met through provision of the method and apparatus pertaining to the warming of a patient described in the following detailed description, particularly when studied in conjunction with the drawings, wherein:

FIG. 1 comprises a flow diagram as configured in accordance with various embodiments of the invention;

FIG. 2 comprises a perspective exploded view as configured in accordance with various embodiments of the invention;

FIG. 3 comprises a perspective view as configured in accordance with various embodiments of the invention;

FIG. 4 comprises a perspective view as configured in accordance with various embodiments of the invention;

FIG. 5 comprises a top plan view as configured in accordance with various embodiments of the invention;

FIG. 6 comprises a side elevational detail schematic view as configured in accordance with various embodiments of the invention;

FIG. 7 comprises a side elevational detail schematic view as configured in accordance with various embodiments of the invention;

FIG. 8 comprises a side elevational detail schematic view as configured in accordance with various embodiments of the invention;

FIG. 9 comprises a block diagram as configured in accordance with various embodiments of the invention; and

FIG. 10 comprises a side elevational schematic view as configured in accordance with various embodiments of the invention.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these teachings a patient warming apparatus includes a compressible patient support pad and an electrically-resistive heating film disposed in conjunction with the compressible patient support pad that is configured to provide heat to at least a portion of a patient that is supported atop the compressible patient support pad. By one approach the electrically-resistive heating film comprises a substantially non-resilient, dead-foldable film substrate.

By one approach the substantially non-resilient, dead-foldable film substrate comprises a polyimide cloth-like film having a conductive coating permanently adhered to one side thereof. This conductive coating can form two physically-separated electrodes. By one approach these electrodes need not be electrically coupled by conductive traces.

These teachings are highly flexible in practice and will accommodate a wide variety of modifications and alterations in practice. By way of example, such a substrate can be located on a patient side of the compressible patient support pad, on a side of the pad that is opposite the patient, and/or within the compressible patient support surface.

So configured, a control circuit can control providing the electrically-resistive heating film with electrical power to thereby control an amount of heat being provided to the patient. By one approach, one or more temperature sensors can serve to provide information to the control circuit regarding any of a variety of useful local temperatures as regards the foregoing.

So configured, a patient warming apparatus having a known, proven form factor and user familiarity significantly avoids physical sensitivities that can lead to a shortened operating lifetime as compared to prior approaches in these same regards. This improved reliability results in a more efficient use of resources and the avoidance of patient-care disruptions that can occur when the patient warming apparatus fails at a time of need.

These and other benefits may become clearer upon making a thorough review and study of the following detailed description. Referring now to the drawings, and in particular to FIG. 1, an illustrative process that is compatible with many of these teachings will now be presented.

At 101 this process 100 provides a compressible patient support pad configured to support at least a portion of a patient (such as, for example, the patient's torso or some significant portion thereof). Referring momentarily to FIG. 2, by one approach this compressible patient support pad 200 can comprise a single such pad 201. By another approach, the compressible patient support pad 200 can additionally comprise a second pad 202 (or additional such layers as desired). When using a plurality of pad layers the various pads may, or may not, have coinciding peripheral dimensions as desired.

Each such pad can be comprised of a material or materials that will provide support to the patient. Accordingly, for many application settings it can be beneficial for one or more such pad layers to comprise a somewhat compressible material, such as a suitable memory foam or other foam, an air mattress, a water mattress, or the like in order to be somewhat giving and accommodating to the shape and comfort of the patient when reclining thereon. The thickness of the compressible patient support pad 200 (and/or any of its constituent layer(s)) can vary as desired and as a function, at least to some extent, of the material employed, the overall dimensions of the compressible patient support pad 200, and the intended use of the completed item (for example, whether the compressible patient support pad 200 is designed for occasional intermittent use or longer-term use by a single patient).

The footprint of the compressible patient support pad 200 can vary as well as desired. In many cases the length and width of the compressible patient support pad 200 will be selected to match the corresponding dimensions of a counterpart patient support platform (such as an operating-room table, a medical-procedures couch, a stretcher, and so forth) with which the compressible patient support pad 200 will be used.

In many cases the compressible patient support pad 200 will include a partial or complete cover (not shown in this view) as well. Such a cover can serve a hygienic and/or aesthetic purpose as desired. Such a cover can also comprise a permanent part of the assembly or can be selectively removable (for example, by use of a zipper, hooks-and-loops fasteners, brads, or the like) as desired.

Generally speaking, a variety of compressible patient support pads are known in the art. The present teachings are not overly sensitive to any particular choices made in these regards. Instead, the present teachings can be successfully employed with essentially any known compressible patient support pad and likely any such pads as are developed in the future.

Referring again to FIG. 1 while also making continued reference to FIG. 2, at 102 the process 100 provides for physically combining the compressible patient support pad 200 with at least one electrically-resistive heating film 203, the latter being configured to provide heat to at least a portion of a patient that is supported atop the compressible patient support pad 200.

By one approach, this electrically-resistive heating film 203 comprises a substantially non-resilient, dead-foldable film substrate. In a classic sense, the expression “dead-foldable” refers to a material that, when folded, will remain in the folded position. Household aluminum foil is an example of a dead-foldable material while thin sheets of paper usually exhibit considerable (but not complete) dead-foldable behavior. Generally speaking, “dead-foldable” in that classic sense refers to materials that will persist the folded state even in the presence of some external forces (such as gravity). Accordingly, a bent piece of aluminum foil will tend to maintain the bend even when lifting and turning the folded foil and exposing the foil to gravitational forces.

To be clear, however, as used herein the expression “dead-foldable” will be understood to also include materials that, when folded, will remain in the folded position in the absence of external forces such as gravity as well as a material that may reform itself readily when exposed to those external forces. A conformable cloth-like material is a useful example in these regards; a towel will remain in a folded state unless and until, say, a corner of the towel is raised and gravity causes the towel to unfold. To some extent as well, the word “non-resilient” serves to highlight this aspect of the electrically-resistive heating film 203.

FIGS. 3 and 4 offer an illustrative example in these regards. In these examples the electrically-resistive heating film 203 is disposed atop and affixed to an underlying compressible pad 201. When an elbow 301 is pressed down on the electrically-resistive heating film 203 as shown in FIG. 3 the underlying pad 201 compresses inwardly and the film 203 tracks that compression at the point of contact with the elbow 301. When this occurs, various folds 302 occur in the electrically-resistive heating film 203.

When the elbow 301 withdraws as shown in FIG. 4, the compressible pad 201 returns to its ordinary uncompressed state. The electrically-resistive heating film 203, being a non-resilient, dead-foldable material per this description, also returns (at least to some substantial extent) to a pre-elbow state that essentially includes few or no folds. The latter occurs not because the electrically-resistive heating film 203 itself is resilient and hence has physical memory regarding that pre-elbow state but rather because the electrically-resistive heating film 203 poses no resistance to the uncompressing pad 201. Accordingly, in this example, the pad 201 acts something like gravity in the example provided above.

By one approach, the substantially non-resilient, dead-foldable film substrate can comprise a polyimide film having an electrically conductive coating permanently adhered to one side (and, by one approach, only one side) thereof. With momentary reference to FIG. 5, and by way of a non-limiting example, the electrically-resistive heating film 203 can comprise a rectangular sheet of the aforementioned polyimide film having two physically-separated and electrically discrete electrodes 502 formed on a same side 501 thereof. In this example the electrically-conductive coating that forms the electrodes 502 can itself comprise, say, a thin layer of silver having a width ranging from about one-fourth of an inch to about three-fourths of an inch. These electrodes 502 can be formed, by one approach, along opposing edges of the electrically-resistive heating film 203.

Other approaches can be considered in these same regards if desired. For example, Eeonix Corporation offers a nanocarbon polymer-coated polyester fiber having a characteristic 36 Ohm resistance per square meter and that is barrier coated for moisture resistance that can serve as the substantially non-resilient, dead-foldable film substrate.

The relative dimensions of the electrically-resistive heating film 203 can of course vary with the application setting. For deployment with a typical operating-room table, this electrically-resistive heating film 203 may have a width of about 19 inches and a length ranging from about 36 inches to about 78 inches as desired. For use with a stretcher pad, a width of about 29 inches and a length of up to 84 inches may be appropriate.

These teachings will accommodate a number of different ways to physically combine the compressible patient support pad 200 with the electrically-resistive heating film 203. Referring again to FIG. 2, when the compressible patient support pad 200 comprises only a single pad 201, by one approach the electrically-resistive heating film 203 can be disposed on a side of the compressible patient support pad 200 that is opposite the patient (i.e., opposite a side/surface of the compressible patient support pad 200 that is nearest the patient during use). By another approach, the electrically-resistive heating film 203 can be disposed on top of the compressible patient support pad 200 as denoted by reference numeral 204 (i.e., on the side/surface of the compressible patient support pad 200 that is nearest the patient during use). Depending upon the materials that comprise the compressible patient support pad 200, the thickness of those materials, and the needs and requirements of the application setting itself, one of these approaches may offer advantages over the other.

These teachings will also support disposing the electrically-resistive heating film 203 within the compressible patient support pad 200. By one approach, and by way of another illustrative example, the electrically-resistive heating film 203 can be placed between two pads 201 and 202 that together comprise the compressible patient support pad 200. By another approach, and by way of yet another illustrative example, the electrically-resistive heating film 203 can be placed within a single pad (for example, by cutting into the pad to create a place for the electrically-resistive heating film 203 to nest within the pad in an installed state).

FIGS. 6-8 provide further details in these regards. These views present the compressible patient support pad 200 in schematic form (and hence the illustrated pad can comprise a single pad or any number of stacked pads as desired) and in combination with a cover 601 that overlies at least the top surface and sides of the compressible patient support pad 200. If desired, of course, the cover 601 can be configured to fully envelope the compressible patient support pad 200.

More particularly, FIG. 6 depicts a physical combination where the compressible patient support pad 200 has the electrically-resistive heating film 203 disposed therein. As illustrated the latter is disposed approximately halfway between the upper surface and bottom surface of the compressible patient support pad 200. These teachings will accommodate locating the electrically-resistive heating film 203 essentially anywhere within the compressible patient support pad 200, including very near the top surface thereof (for example, within, say, 0.5 inches) or very near the bottom surface thereof.

FIG. 6 also depicts the electrically-resistive heating film 203 as being disposed substantially co-planar with the compressible patient support pad 200. Again, if desired, these teachings will accommodate other approaches in these regards. By one approach, for example, the electrically-resistive heating film 203 can be positioned at a different angle, such as fifteen degrees or thirty degrees, with respect to the compressible patient support pad 200.

FIG. 7 depicts a physical combination where the electrically-resistive heating film 203 is disposed on the patient side of the compressible patient support pad 200. In this illustrative example the cover 601 overlies the electrically-resistive heating film 203.

And FIG. 8 depicts a physical combination where the electrically-resistive heating film 203 is located on the underside of the compressible patient support pad 200 and hence on an opposite side of the side of the compressible patient support pad 200 that is closest to the patient during use. In this particular illustrated example the cover 601 does not envelope the electrically-resistive heating film 203. If desired, however, the cover 601 can be configured to cover the electrically-resistive heating film 203.

These teachings will accommodate a variety of ways to attach the electrically-resistive heating film 203 to the compressible patient support pad 200. By one approach the one can be sewn to the other. By another approach a permanent adhesive can serve in these regards. By yet another approach the compressible patient support pad 200 can include a pocket formed on an outer surface that is sized and configured to snuggly receive the electrically-resistive heating film 203. These teachings will also accommodate using any of a variety of temporary affixment mechanisms including but not limited to hooks-and-loops fasteners, snaps, zippers, magnets, and so forth as desired.

Referring to FIGS. 1 and 9, by one optional approach and at 103 this process 100 will accommodate providing one or more temperature sensors 902. This temperature sensor 902 can serve to detect a local temperature of interest such as a local temperature as corresponds to a point of contact between the compressible patient support pad 200 and the patient or a surface on or near the electrically-resistive heating film 203 itself.

A variety of temperature sensors are known in the art and will serve well in these regards. A variety of known methodologies also exist to communicate sensed temperature information from such a temperature sensor 902 to another location. Examples in these regards include but are not limited to a variety of wireless and non-wireless approaches. As the present teachings are not overly sensitive to any particular selections in these regards, further elaboration will not be provided here for the sake of brevity.

The temperature information obtained with this temperature sensor 902 can be displayed to keep medical services providers in the vicinity of the patient informed as to the monitored temperature of interest. As will be described below, this temperature information can also serve to help utilize the electrically-resistive heating film 203 to appropriately and automatically warm a patient.

At 104 the process 100 provides a control circuit 901. Such a control circuit 901 can comprise a fixed-purpose hard-wired platform or can comprise a partially or wholly programmable platform. These architectural options are well known and understood in the art and require no further description here. This control circuit 901 is configured (for example, by using corresponding programming as will be well understood by those skilled in the art) to carry out one or more of the steps, actions, and/or functions described herein.

This control circuit 901 can operably couple to one or more of the aforementioned electrically-resistive heating films 203 (and also to one or more of the aforementioned temperature sensors 902 as desired). In this illustrative example the control circuit 901 is configured to control an amount of electrical current being provided to the electrically-resistive heating film 203. Accordingly, the control circuit 901 controls an amount of heat being provided to a corresponding patient by the electrically-resistive heating film 203. When operably coupled to one or more temperature sensors 902, the control circuit 901 can be configured to control the provisioning of electrical power to the electrically-resistive heating film 203 as a function, at least in part, of temperature information provided by the temperature sensor 902.

FIG. 10 provides an illustrative example in these regards. No particular limitations are intended by way of the specifics of this example.

In this example a single electrically-resistive heating film 203 is disposed on the patient side of a patient-supporting pad 201 and a cover 601 overlies that assembly. The electrically-resistive heating film 203 is smaller than the pad's upper surface and is positioned to underlie, to at least a considerable extent, the torso of a patient 1001 who lies in ordinary course and as expected atop the compressible patient support pad 200. Also in this example two temperature sensors 902 are positioned within the pad 201 and relatively proximal to portions of the patient 1001 that are to be warmed by the electrically-resistive heating film 203. (These temperature sensors 902 couple to a control circuit (not shown in this figure) as described above, as does the electrically-resistive heating film 203.)

So configured, the control circuit 901 controls the provision of electrical power to the electrically-resistive heating film 203 to warm the patient 1001 in a desired manner. (Numerous heating profiles and approaches are known and the present teachings are not particularly sensitive to the selection of any particular methodology in these regards. Accordingly, for the sake of brevity, further discussion regarding the specifics of how the electrically-resistive heating film 203 is specifically used to warm a given patient in a given application setting need not be presented here.)

These teachings are highly flexible in practice and will accommodate a variety of modifications to the foregoing description. As but one example in these regards, a given compressible patient support pad 200 can be physically combined with a plurality of electrically-resistive heating films 203. That plurality of electrically-resistive heating films 203 can be disposed substantially coplanar to one another or not as desired. Such a plurality of electrically-resistive heating films 203 can also, if desired, be disposed in partial or complete vertical registration with one another.

So configured, the resultant patient warming apparatus is considerably less susceptible to eventual failure as a result of compromises to the physical integrity of the heating element due to any of a variety of forces acting upon the heating element. Accordingly, such an apparatus is better suited to the daily use, storage, deployment, and cleaning regimens of a typical medical-services facility.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

We claim:

1. A patient warming apparatus suitable for use with a patient support platform, the patient warming apparatus comprising:
a compressible patient support pad configured to support at least a portion of a patient;
an electrically-resistive heating film disposed in conjunction with the compressible patient support pad and configured to provide heat to at least a portion of a patient that is supported atop the compressible patient support pad, the electrically-resistive heating film comprising a substantially non-resilient, dead-foldable film substrate.

2. The patient warming apparatus of claim 1 wherein the substantially non-resilient, dead-foldable film substrate comprises a polyimide film.

3. The patient warming apparatus of claim 1 wherein the substantially non-resilient, dead-foldable film substrate has a conductive coating permanently adhered to one side thereof.

4. The patient warming apparatus of claim 3 wherein the conductive coating is disposed on only the one side of the substantially non-resilient, dead-foldable film substrate.

5. The patient warming apparatus of claim 3 wherein the conductive coating comprises two physically-separated electrodes.

6. The patient warming apparatus of claim 5 wherein the two physically-separated electrodes are not electrically coupled by any conductive traces.

7. The patient warming apparatus of claim 3 wherein the conductive coating comprises silver.

8. The patient warming apparatus of claim 1 wherein the electrically-resistive heating film is disposed within the compressible patient support pad.

9. The patient warming apparatus of claim 1 wherein the electrically-resistive heating film is disposed on a side of the compressible patient support pad that is opposite the patient.

10. The patient warming apparatus of claim 1 further comprising:
a control circuit configured to control providing the electrically-resistive heating film with electrical power to thereby control an amount of heat being provided to the patient.

11. The patient warming apparatus of claim 10 wherein the control circuit is configured to control an amount of electrical current being provided to the electrically-resistive heating film.

12. The patient warming apparatus of claim 10 further comprising:
at least one temperature sensor that operably couples to the control circuit;
wherein the control circuit is configured to control providing the electrically-resistive heating film with electrical power as a function, at least in part, of temperature information provided by the temperature sensor.

13. A method comprising:
providing a compressible patient support pad configured to support at least a portion of a patient;
physically combining the compressible patient support pad with an electrically-resistive heating film that is configured to provide heat to at least a portion of a patient that is supported atop the compressible patient support pad, the electrically-resistive heating film comprising a substantially non-resilient, dead-foldable film substrate.

14. The method of claim 13 wherein the substantially non-resilient, dead-foldable film substrate comprises a polyimide film.

15. The method of claim 13 wherein the substantially non-resilient, dead-foldable film substrate has a conductive coating permanently adhered to one side thereof.

16. The method of claim 15 wherein the conductive coating is disposed on only the one side of the substantially non-resilient, dead-foldable film substrate.

17. The method of claim 15 wherein the conductive coating comprises two physically-separated electrodes.

18. The method of claim 17 wherein the two physically-separated electrodes are not electrically coupled by any conductive traces.

19. The method of claim 13 further comprising:
providing a control circuit configured to control providing the electrically-resistive heating film with electrical power to thereby control an amount of heat being provided to the patient.

20. The method of claim 19 further comprising:
providing at least one temperature sensor that operably couples to the control circuit; wherein the control circuit is configured to control providing the electrically-resistive heating film with electrical power as a function, at least in part, of temperature information provided by the temperature sensor.
